(19) United States
(12) Patent Application Publication
(10) Pub. No.: US 2002/0048532 A1
(43) Pub. Date: Apr. 25, 2002

(54) Disposable electrode for whole blood hemoglobin (HGB) and hematocrit (HCT) measurement, and preparation and application thereof

(76) Inventors: Yueh-Hui Lin, Yun Lin Hsien (TW); Thomas Y.S. Shen, Hsinchu (TW)

Correspondence Address: ROTHWELL, FIGG, ERNST & KURZ, P.C., 555 13TH Street , N.W., Washington, DC, 20004, US

(21) Appl. No.: 09/771,634

(22) Filed: Jan. 30, 2001

(30) Foreign Application Priority Data
Sep. 1, 2000 (TW) 89117880

(51) Int. Cl. G01N 33/49

(52) U.S. Cl. 422/82.01; 436/66; 422/73

(57) ABSTRACT

The subject invention relates to a disposable hemoglobin and hematocrit detecting electrode strip, the preparation and the use thereof. The concentration of hemoglobin and hematocrit in a liquid sample can be determined by electrochemically analyzing the liquid sample under a low operation voltage of below 400 mV. When the electrode strip is applied to detect the concentration of hemoglobin and hematocrit in human body, the whole blood is directly used as the sample. The hemoglobin and hematocrit detecting electrode strip is modified by a water-soluble redox electron mediator. The electrode strip simplifies the analysis of hemoglobin and hematocrit, is conveniently portable and can be easily manufactured in mass-production.

### FIELD OF THE INVENTION

The subject invention relates to an electrode strip which can easily determine the concentration of hemoglobin and hematocrit in a liquid sample, and to the preparation and the applications thereof. More specifically, the invention relates to a disposable hemoglobin and hematocrit electrode strip based on the theory of electrochemistry modified by a water-soluble redox electron mediator, which is disposable, and suitable for household use, screening for blood banks. In addition, said electrode strip can precisely detect the concentration of hemoglobin and hematocrit without any interference caused by other components in a liquid.

### BACKGROUND OF THE INVENTION

Hemoglobin is also called hematochrome, which is 90% of erythrocyte and is composed of four globins and four ferrohemes. The main function of hemoglobin is to transport oxygen and carbon dioxide. The decrease of the concentration of hemoglobin shows anemia; and the increase shows polycythemia. Many methods can be used to determine the concentration of hemoglobin, including: (1) chemical method; (2) gas determination method; (3) specific density method; and (4) colorimetric method. The drawback of the former three methods are respectively: (1) being troublesome and time-consuming, (2) requiring specific equipment and (3) providing poor preciseness. Therefore, those methods are not useful in routine tests. Normally, routine test is performed by colorimetric method. In general, the colorimetric method includes acid hematin method and cyanmet-hemoglobin method. The acid hematin method: Sahli's method is normally used, which utilizes Sahli's tube to suck up 0.2 ml of HCl (0.1N) to mix with 0.1 ml of blood for hemolysis and reaction for 15 minutes. Water is dropwise added to the solution until the color of the solution and the color standards are consistent while the color of the solution is colorimetered with the color standards of the Sahli-Hellige hemoglobinometer. Because the color is subjectively judged by the operator in this method, the CV % is larger (about 5% to 10%). The cyanmet-hemoglobin method: the Drabkin's solution is used to dissolve the erytirocyte, and the component of the Drabkin's solution, K₃Fe(CN)₆, can oxidize the ferrous moiety of hemoglobin into ferric moiety to form ferriheme (met-Hb; MHb). Ferriheme may combine with potassium cyanide (KCN) to form stable ferriheme cyanide, then ferriheme cyanide is colorimetered at 540 nm with a photoelectric colorimeter. This method is rapid and precise, and is publicly recognized as a standard method. Except that sulfhemoglobin does not show reaction, all types of hemoglobin can be detected. In this method, there is a hypertoxic pollution problem caused by potassium cyanide.

Hematocrit (Hct) is referred to the percentage of globin volume, which is used for determining the proportion of erythrocyte contained in a certain amount of blood. Hematocrit is a good index for the judgment of anemia, which can correct the detection of erythrocyte and hemoglobin and determine the RBC indices. The method for determination of hematocrit includes centrifugation method and automatic globin counting method. The centrifugation method comprises precipitating the whole blood under certain centrifugation force by use of Wintrobe's tube or capillary tube (75*1 mm) to obtain three layers, i.e., a blood plasma layer, a gray-yellow layer and an erythrocyte layer, respectively. The volume percentage of the erythrocyte accumulated is determined and converted to hematocrit. The former method utilizing the Wintrobe's tube is the standard method for determination of hematocrit, but a large amount of blood is required. On the other hand, the latter method utilizing the capillary tube must be performed in combination with the use of a centrifugal machine and a hematocrit proportion ruler and need a high operation technique, although a little amount of blood is required. The automatic globin counting method is based on that the impulse is varied with the globin particles passing through the electric field in an electrolyte solution, and thus the cell number and cell size can be determined thereby. The other way of the automatic globin counting method is based on the cell number converted from the light of flash generated by the light beam when the particles of globin pass through tiny channels. Because the automatic globin counting method requires an impulse or a light of flash analyzer and a large amount of an electrolyte solution, and the analyzer need to be maintained and corrected, this method cannot be conveniently utilized.

Therefore, there is a need for a more precise and easy method so as to determine the concentration of erythrocyte and hemoglobin.

### SUMMARY OF THE INVENTION

An object of the subject invention is to provide a disposable hemoglobin and hematocrit detecting electrode strip which can directly analyze the concentration of hemoglobin and hematocrit in a blood sample by use of the electrode strip operated under a low operation voltage of below 400 mV and a pH value from 5.0 to 8.0.

Another object of the subject invention is to provide a simplified method for producing disposable hemoglobin and hematocrit detecting electrode strip, which does not require any bioactive substances and thus simplifies and improve the efficiency of the production procedures. This method reduces the manufacturing costs and can rapidly proceed by screen printing on a large scale so that the produced electrode strip can be easily provided for the people to use.

A further object of the subject invention is to provide an equipment and a method for rapid, convenient and safe detection of hemoglobin and hematocrit in a blood sample. The blood sample is directly dropped on the disposable hemoglobin and hematocrit detecting electrode strip and the concentration of hemoglobin in the blood sample can be easily detected via a redox reaction. The hematocrit value can be obtained in accordance with the positive correlation between the concentration of hemoglobin and the hematocrit value.

### BRIEF DESCRIPTION OF THE DRAWINGS

FIG. 1a is a top view diagram of an electrode strip of the subject invention.

FIG. 1b is a front view diagram of an electrode strip of the subject invention.

FIGS. 2a to 2d show an embodiment of the preparation of the electrode strip of the invention. FIG. 2 shows the screen-printing on an electric insulating substrate 1 a conducting film 2 containing at least an anode and a cathode. FIG. 2b shows the screen-printing on the conducting film a partially electric insulating film 3, and a part of the conducting film is uncovered to form an anode connector 7, a cathode connector 6, a working electrode 8 and a reference electrode 9. FIG. 2c shows the screen-printing of a carrier and a conductive mediator on a region of a reaction film 4 formed by the working electrode 8 and the reference electrode 9. FIG. 2d shows the coating of the dried reaction film on a protection film 5.

FIG. 3 is a graph showing comparative results of the concentration of hemoglobin in the same whole blood sample analyzed by the detecting electrode strip of the subject invention and the SIGMA hemoglobin testing kit.

FIG. 4 is a graph showing comparative results of the hematocrit in the same sample analyzed by the detecting electrode strip of the subject invention and the capillary micro-amount hemoglobin percentage method.

### DETAILED DESCRIPTION OF THE INVENTION

The subject invention relates to a detecting electrode strip modified by a water-soluble redox electron mediator. The detecting electrode strip applied to a detecting system of low operation voltage of below 400 mV can sensitively and specifically detect the concentration of hemoglobin in liquid by catching signals generated by a redox current of a redox electron mediator and hemoglobin without interference caused by other components in the liquid. In particular, the hemoglobin detecting electrode strip of the subject invention, based on the theory of electrochemistry, can directly detect the concentration of hemoglobin in a blood sample. On account of the positive correlation between the concentration of hemoglobin and hematocrit, the hematocrit value can be calculated from the concentration of hemoglobin.

The subject invention provides a disposable hemoglobin and hematocrit detecting electrode strip, which comprises:

(a) an electric insulating substrate;

(b) a conducting film coated on one side of the electric insulating substrate to form isolated and disconnected an anode and a cathode;

(c) an electric insulating film coated on a part of the conducting film, wherein one end of an uncovered anode of the conducting film forms at least a reference electrode and the other end an anode connector, and one end of an uncovered cathode of the conducting film forms at least a working electrode and the other end a cathode connector; and

(d) a reaction film comprising a carrier, a surfactant and a conductive mediator, being screen printed on a region containing at least a working electrode and a reference electrode so as to connect the working electrode and the reference electrode individually, wherein the carrier comprises a microcrystalline cellulose, a polymer and a buffer solution; the surfactant comprises an amphoteric substance having a hydrophobic moiety and a hydrophilic moiety; and the conductive mediator comprises an electrolyte with a lower redox potential than that of hemoglobin.

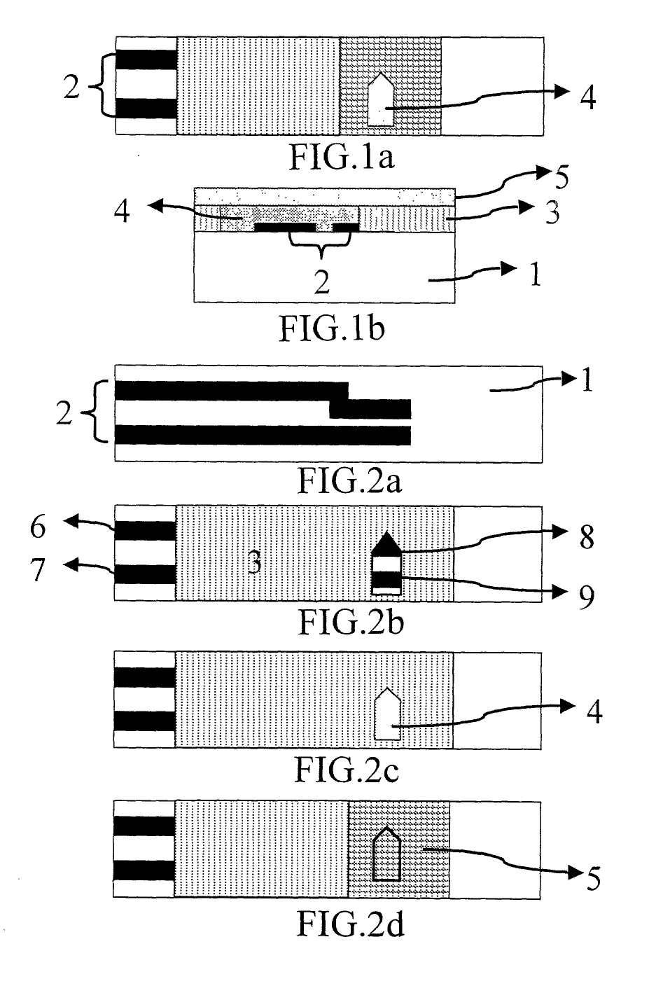

According to the hemoglobin and hematocrit detecting electrode strip of the subject invention, the appearance of an embodiment is shown in FIGS. 1a and 1b. From the figures, the electrode strip is a rectangular film shape. FIGS. 1a and 1b are respectively a top view diagram and a front view diagram of the hemoglobin and hematocrit detecting electrode strip of the subject invention. The structure of the electrode strip comprises an electric insulating substrate 1, a conducting film 2 coated on the insulating substrate, an electric insulating film 3 coated on a part of the conducting film 2, and a reaction film 4 for reacting with a sample.

The electric insulating substrate suitable for the subject invention has a flat surface as well as an insulation property and is thermal-resistant from 40° C. to 80° C. for thermal processing and increasing the conductivity and adherence of the conducting film 2. The materials suitable for the electric insulating substrate are selected from the group consisting of polyvinyl chloride (PVC), fiber glass (FR-4), polyester suphone, bakelite plate, polyethylene terephthalate (PET) plate, polycarbonate (PC) plate, glass plate and ceramics plate (CEM-1).

The conducting film 2 of the electrode strip comprises a set of isolated, disconnected and symmetric anode and cathode so as to connect with an amperometric sensor. The cathode is partially covered by the electric insulating film 3 and two uncovered ends of the cathode are a working electrode and a cathode connector, respectively. The working electrode of the cathode is then covered by the reaction film 4 and is used to detect an induced electric effect of samples during the electrochemical reaction of hemoglobin and hematocrit. The cathode connector is used to connect with an amperometric sensor. The anode is also partially covered by the electric insulating film 3 and two uncovered ends of the anode are a reference electrode and an anode connector, respectively. The reference electrode of the anode is covered by the reaction film 4 and cooperates together with the working electrode of the cathode to detect the induced electric effect. The anode connector is also used to connect with the amperometric sensor. The material suitable for the conducting film is a conductive slurry material suitable for screen printing, such as carbon ink, gold ink, silver ink, the mixture of carbon and silver ink, volatile graphite, copper ink, or the mixture of the above (for example, printing silver ink first and then printing carbon ink).

According to the hemoglobin and hematocrit detecting electrode strip of the subject invention, the electric insulating film 3 is coated on the same surface of the conductive film 2 of the electric insulating substrate 1, but does not cover the cathode connector, anode connector, working electrode and reference electrode. The suitable thickness of the electric insulating film is preferably 0.6 mm or above. The region uncovered by the electric insulating film 3 includes the working electrode and the reference electrode and forms a reaction region which is then coated by the reaction film 4 for testing samples.

The reaction film 4 of the electrode strip of the subject invention comprises a carrier, a surfactant and a conductive mediator. The carrier is a slurry material suitable for screen printing and comprises a microcrystalline cellulose, a polymer and a buffer solution. The microcrystalline cellulose is used to absorb a sample because the sample is hydrophilic and difficult to attach to a hydrophobic conducting film. The microcrystalline cellulose enhances the absorption of sample and enhances the signals to be transferred to the conducting film. For mass-production, microcrystalline cellulose of the carrier has a size of preferably below 100 μm. The microcrystalline cellulose thus can be distributed all over the reaction film area and ensure the signals be thoroughly transferred. The amount of the microcrystalline cellulose of the carrier ranges from about 0% to about 25% by weight of the reaction film.

The polymer of the carrier is selected from the group consisting of polyvinyl alcohol (PVA), polyvinylpyrrolidone (PVP), polyethylene glycol (PEG), inkatin, carboxymethyl cellulose (CMC), methyl cellulose and the mixture thereof. The amount of the polymer of the carrier ranges from about 0% to about 40% by weight of the reaction film. The polymer is to facilitate the reaction film having a certain viscosity for screen printing. The reaction film can therefore be distributed thoroughly.

The buffer solution of the carrier is selected from the group consisting of potassium dihydrogen phosphate, dipotassium orthophosphate, salts of boric acid, citric acid, Tris or the mixtures thereof. The amount of the buffer solution of the carrier is preferably 0% to 6% by weight of the reaction film. The buffer solution is used to adjust the pH value of from 5.0 to 8.0 for a reaction between the reaction film and hemoglobin and hematocrit.

The other components of the reaction film are a surfactant and a conductive mediator. The surfactant is used to enhance the reactivity of hemoglobin and the conductive mediator, which can be any surfactant known to persons skilled in the art. The conductive mediator comprises an electrolyte with a lower redox potential than that of hemoglobin. The conductive mediator will change from oxidation state to reduction state after reacting with hemoglobin. The conductive mediator can then reverse to oxidation state by applying a forced voltage. The changes of, such as potential, resistance, or current caused by electrochemical reaction, could be transferred through the conducting film, i.e. from the working electrode and the reference electrode connected with the reaction film to the cathode connector and the anode connector. The conductive mediator suitable for the reaction film is preferably a water-soluble redox conductive mediator. One embodiment of the invention illustrates the use of Triton X-100 as a surfactant and that of potassium ferricyanide as a conductive mediator. The amount of the conductive mediator of the reaction film preferably ranges from 2% to 10% by weight of the reaction film.

In accordance with the subject invention, the reaction film 4 of the electrode strip is optionally coated with a protection film 5 to protect the reaction film.

In accordance with the hemoglobin and hematocrit detecting electrode strip of the invention, it is not necessary to use the Drabkin's solution comprising potassium cyanide, which can avoid the hypertoxic pollution and the difficulty in the preservation of the Drabkin's solution, and improve the convenience and reduce the mental burden of the operator on such hypertoxic reagent and the environmental pollution. While determining the hematocrit, there is no need to use a centrifugal machine or an automatic globin counting equipment, thus the convenience is significantly improved.

The hemoglobin detecting electrode strip of the invention can simplifies the manufacture process, wherein a carrier and a conductive mediator can be attached on the electric insulting substrate in one step and reduces the production cost. Because the manufacture procedure is simple and the detecting electrode strip does not contain bio-reactive substances, high sensitivity requirement of the manufacture process can be achieved and the production cost can be decreased.

Therefore, the subject invention further provides a process for producing a disposable hemoglobin and hematocrit detecting electrode strip, which comprises:

(a) coating a conducting film on one side of an electric insulating substrate and forming an isolated and disconnected anode and an isolated and disconnected cathode;

(b) coating an electric insulating film on a part of the conducting film, wherein one end of an uncovered anode of the conducting film is at least a reference electrode and the other end of an anode connector, and one end of an uncovered cathode of the conducting film is at least a working electrode and the other end a cathode connector; and

(c) attaching a reaction film to an electric insulating substrate, wherein the reaction film cover a region containing at least the working electrode and the reference electrode so as to connect the working electrode and the reference electrode individually, and wherein the reaction film comprises a carrier, a surfactant and a conductive mediator; the carrier comprises a microcrystalline cellulose, a polymer and a buffer solution; the surfactant comprises an amphoteric substance having a hydrophobic moiety and a hydrophilic moiety; and the conductive mediator comprises an electrolyte with a lower redox potential than that of hemoglobin.

According to step (a) of the process for producing a disposable hemoglobin and hematocrit detecting electrode strip of the subject invention, a conducting film is first coated on one side of a flat substrate to form at least an anode and a cathode which are separately isolated from each other. The conducting film is preferably screen printed on the substrate comprising an anode and a cathode, as shown in FIG. 2a.

According to step (b) of the process of the invention, an electric insulating film with a thickness of 0.6 mm or above is partially printed onto the conducting film. The uncovered parts of the conducting film form a cathode connector 6, an anode connector 7, a working electrode 8 and a reference electrode 9, as shown in FIG. 2b. An area formed by the working electrode 8 and the reference electrode 9 in a circle or any other suitable shape is an area of reaction film.

According to step (c) of the process of the invention, the conductive slurry materials containing a microcrystalline cellulose, a polymer, a buffer solution, a conductive mediator and a surfactant are coated on the arrow area of the reaction film 4, as shown in FIG. 2c. The conductive slurry materials are preferably screen printed on the area of the reaction film.

The process of the subject invention can further comprise drying the above reaction film at 40° C. to 80° C. A protection film is optionally coated on and around the circle area of the reaction film 4, as shown in FIG. 2d. The disposable hemoglobin and hematocrit detecting electrode strip is thereby produced.

Several conventional screen printing techniques can be used in the process for producing the disposable hemoglobin and hematocrit detecting electrode strip of the subject invention. Moreover, a new screen printing technology disclosed by one of the inventors of the subject invention and contained in R.O.C. patent application Ser. No. 85,109,554 could also be applied in the production method of the subject invention.

The detecting method of the electrode strip of the subject invention can proceed easily by using an electrochemical detecting equipment. The reaction current caused by the redox reaction of hemoglobin and hematocrit can be detected in a hemoglobin and hematocrit detecting equipment by dropping a whole-blood sample on the reaction area of the hemoglobin and hematocrit detecting electrode strip of the subject invention. Such an electrochemical reaction technology is commonly applied in electrochemical blood sugar monitor for detecting blood sugar. When the electrode strip of the subject invention is connected to a sensor, the sensor can apply an output voltage to the electrode strip generated by a voltage output device, and a signal receiver receives a current, voltage or resistance change generated by the above chemical reaction and transmits the signals to the display equipment to demonstrate the concentration of hemoglobin or the hematocrit value. The method of directly detecting hemoglobin and hematocrit in whole-blood is novel and first disclosed in the subject invention by using a redox electron mediator to transfer signals of the redox reaction of hemoglobin and hematocrit and by controlling the reaction at pH value from 5.0 to 8.0 as well as a low operation voltage of below 400 mV.

Therefore, the subject invention also provides a hemoglobin and hematocrit detecting equipment which comprises a disposable hemoglobin and hematocrit detecting electrode strip and an amperometric sensor, which can directly analyze the concentration of hemoglobin and hematocrit in blood. The detecting equipment comprises:

(A) a disposable hemoglobin and hematocrit detecting electrode strip, said strip comprises:

(a) an electric insulating substrate;

(b) a conducting film coated on one side of the electric insulating substrate to form isolated and disconnected an anode and a cathode;

(c) an electric insulating film coated on a part of the conducting film, wherein one end of an uncovered anode of the conducting film forms at least a reference electrode and the other end an anode connector, and one end of an uncovered cathode of the conducting film forms at least a working electrode and the other end a cathode connector; and

(d) a reaction film comprising a carrier, a surfactant and a conductive mediator, being screen printed on a region containing at least the working electrode and the reference electrode so as to connect the working electrode and the reference electrode individually, wherein the carrier comprises a microcrystalline cellulose, a polymer and a buffer solution; the surfactant comprises an amphoteric substance having a hydrophobic moiety and a hydrophilic moiety; and the conductive mediator comprises an electrolyte with a lower redox potential than that of hemoglobin; and

(B) an amperometric sensor, which comprises a voltage output equipment, a signal receiver and a display equipment;

wherein the amperometric sensor is connected to the anode connector and the cathode connector of the hemoglobin and hematocrit detecting electrode strip; the voltage output equipment provides a voltage of below 400 mV to the reaction film of the hemoglobin and hematocrit detecting electrode strip so as to oxidize the conductive mediator from reduction state to oxidation state after being reacted with hemoglobin and hematocrit of the sample; the signal receiver receives a current, voltage or resistance change generated during a redox reaction and transmits the change to the display equipment to demonstrate the concentration of hemoglobin and the hematocrit value in the sample.

In addition, the subject invention also provides a method for the detection of hemoglobin and hematocrit, which comprises dropping the liquid sample to the disposable hemoglobin and hematocrit detecting electrode strip of the invention, controlling the reaction of hemoglobin and hematocrit at a low operation voltage of below 400 mV and a pH value from 5.0 to 8.0, and determining the concentration of hemoglobin and the hematocrit value in liquid sample. This method can avoid interference caused by the simultaneous oxidation of other components in the liquid sample which are easily to be oxidized, and thus can precisely determine the concentration of hemoglobin and the hematocrit value in the blood sample.

The following examples are exemplified to describe in detail the subject invention but not to confine the subject invention.

### EXAMPLE

Example 1

On one flat side of polyvinyl chlorine (PVC) substrate, carbon ink was screen printed to form a conducting film 2 comprising a set of isolated and disconnected anode and cathode and then dried at 40° C. to 80° C. An electric insulating film 3 with a thickness of about 0.6 mm was subsequently screen printed on the conducting film 2 partially. The uncovered part of the conducting film formed a cathode connector 6, an anode connector 7, a working electrode 8 and a reference electrode 9. The circle area formed by working electrode 8 and reference electrode 9 was an area of reaction film 4.

The slurry materials comprising the following components and proportions were then screen printed on the arrow area of reaction film 4.

| | |
|---|---|
| Microcrystalline cellulose (diameter: average about 20 μm) | 21.2% |
| PEG, polyethylene glycol | 0.3% |
| PVP, polyvinylpyrrolidone | 13.4% |
| K₂HPO₄ | 0.04% |
| KH₂PO₄ | ∴0.1% |
| H₂O | 56.96% |
| Potassium ferricyanide | 5% |
| Triton X-100 | 3% |

After screen printing the reaction film 4, it was dried at 40° C. to 80° C. The arrow area of the reaction film 4 on the electric insulating film 3 was coated with glue. A protection film 5 was coated on and around the arrow area of reaction film 4. The disposable hemoglobin and hematocrit detecting electrode strip was obtained.

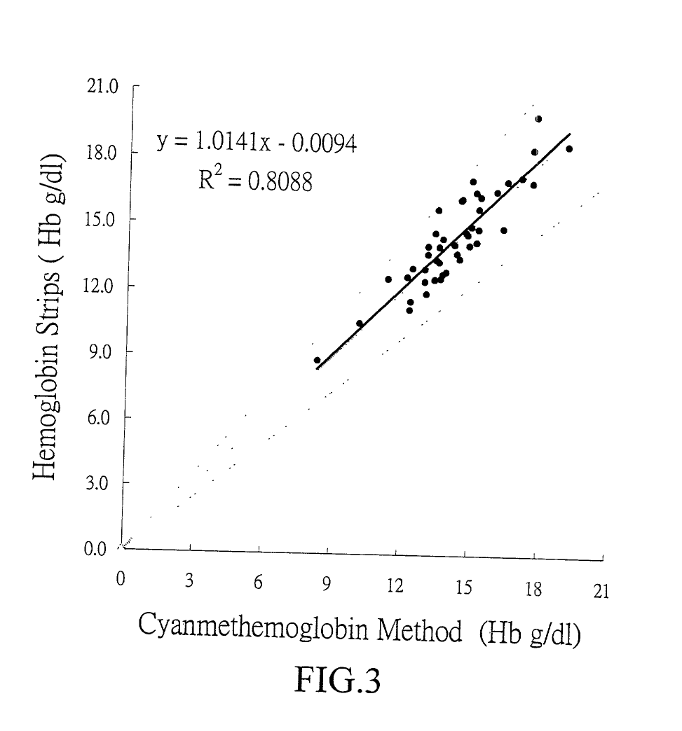

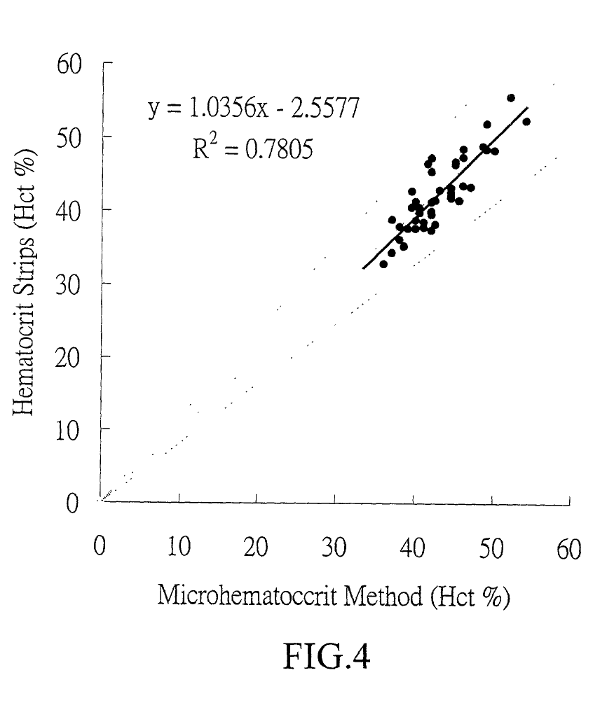

The disposable hemoglobin and hematocrit detecting electrode strip obtained from the above was used to determine the concentration of hemoglobin and hematocrit in a whole-blood sample. It was found that the concentration of hemoglobin determined by the disposable hemoglobin and hematocrit detecting electrode strip of the invention is the same as that detected by the conventional method. FIG. 3 shows the comparative results of the concentration of hemoglobin in whole-blood sample determined by the invention and the SIGMA hemoglobin detection kit; and FIG. 4 shows the comparative results of the hematocrit in whole-blood sample determined by the invention and the capillary micro-amount hemoglobin percentage method. The results show that the disposable hemoglobin and hematocrit detecting electrode strip of the invention can precisely determine the concentration of hemoglobin and the hematocrit value in a blood sample.

Example 2

The procedures described in Example 1 were repeatedly operated, except that the components and proportions of the slurry materials were changed as follows:

| | |
|---|---|
| Microcrystalline cellulose (diameter: average about 20 μm) | 2.2% |
| PEG, polyethylene glycol | 19.8% |
| K₂HPO₄ | 0.7% |
| Citric acid | 1.5% |
| H₂O | 67.8% |
| Potassium ferricyanide | 4% |
| Triton X-100 | 4% |

The disposable hemoglobin and hematocrit detecting electrode strip obtained from the above was used to determine the concentration of hemoglobin and hematocrit in a whole-blood sample. It was found that the concentration of hemoglobin determined by the disposable hemoglobin and hematocrit detecting electrode strip of the invention is the same as that detected by the conventional method. It shows that the disposable hemoglobin and hematocrit detecting electrode strip of the invention can precisely determine the concentration of hemoglobin and the hematocrit value in a blood sample.

Example 3

The procedures described in Example 1 were repeatedly operated, except that the components and proportions of the slurry materials were changed as follows:

| | |
|---|---|
| Microcrystalline cellulose (diameter: average about 20 μm) | 25% |
| PVA, polyvinyl alcohol | 13% |
| PVP, polyvinylpyrrolidone | 7% |
| K₂HPO₄ | 0.7% |
| H₂O | 42.3% |
| Potassium ferricyanide | 7% |
| Triton X-100 | 5% |

The disposable hemoglobin and hematocrit detecting electrode strip obtained from the above was used to determine the concentration of hemoglobin and hematocrit in a whole-blood sample. It was found that the concentration of hemoglobin determined by the disposable hemoglobin and hematocrit detecting electrode strip of the invention is the same as that detected by the conventional method. It shows that the disposable hemoglobin and hematocrit detecting electrode strip of the invention can precisely determine the concentration of hemoglobin and the hematocrit value in a blood sample.

Example 4

The procedures described in Example 1 were repeatedly operated, except that the components and proportions of the slurry materials were changed as follows:

| | |
|---|---|
| Microcrystalline cellulose (diameter: average about 20 μm) | 5.6% |
| H₂O | 89.4% |
| Potassium ferricyanide | 3% |
| Triton X-100 | 2% |

The disposable hemoglobin and hematocrit detecting electrode strip obtained from the above was used to determine the concentration of hemoglobin and hematocrit in a whole-blood sample. It was found that the concentration of hemoglobin determined by the disposable hemoglobin and hematocrit detecting electrode strip of the invention is the same as that detected by the conventional method. It shows that the disposable hemoglobin and hematocrit detecting electrode strip of the invention can precisely determine the concentration of hemoglobin and the hematocrit value in a blood sample.

From the examples mentioned hereinabove, it is clear that the disposable hemoglobin and hematocrit detecting electrode strip of the subject invention can be prepared by simple procedures. The detecting electrode strip can directly utilize the whole-blood sample to precisely analyze the concentration of hemoglobin and obtain the hematocrit value by controlling the reaction on the electrode at a low operation voltage of below 400 mV and a pH value from 5.0 to 8.0.

With the disclosed invention, apparently numerous modifications and variations can be made without departing from the scope and spirit of this the subject invention. Therefore the subject invention is intended to be limited only as indicated in the following claims.

We claim:

1. A disposable hemoglobin and hematocrit detecting electrode strip which comprises:
(a) an electric insulating substrate;
(b) a conducting film coated on one side of the electric insulating substrate to form isolated and disconnected an anode and a cathode;
(c) an electric insulating film coated on a part of the conducting film, wherein one end of an uncovered anode of the conducting film forms at least a reference electrode and the other end an anode connector, and one end of an uncovered cathode of the conducting film forms at least a working electrode and the other end a cathode connector; and
(d) a reaction film comprising a carrier, a surfactant and a conductive mediator, being screen printed on a region containing at least the working electrode and the reference electrode so as to connect the working electrode and the reference electrode individually, wherein the carrier comprises a microcrystalline cellulose, a polymer and a buffer solution; the surfactant comprises an amphoteric substance having a hydrophobic moiety and a hydrophilic moiety; and the conductive mediator comprises an electrolyte with a lower redox potential than that of hemoglobin.

2. The detecting electrode strip according to claim 1, wherein the thickness of the electric insulating film is 0.6 mm or above.

3. The detecting electrode strip according to claim 1, wherein the carrier, the surfactant and the conductive mediator are formulated to a slurry material suitable for screen printing.

4. The detecting electrode strip according to claim 1, wherein the microcrystalline cellulose of the carrier has a size below 100 μm.

5. The detecting electrode strip according to claim 1, wherein the amount of the microcrystalline cellulose of the carrier ranges from about 0% to about 25% by weight of the reaction film.

6. The detecting electrode strip according to claim 1, wherein the polymer of the carrier is selected from the group consisting of polyvinyl alcohol (PVA), polyvinylpyrrolidone (PVP), polyethylene glycol (PEG), inkatin, carboxymethyl cellulose (CMC), methyl cellulose and the mixture thereof.

7. The detecting electrode strip according to claim 1, wherein the amount of the polymer of the carrier ranges from about 0% to about 40% by weight of the reaction film.

8. The detecting electrode strip according to claim 1, wherein the buffer solution of the carrier comprises potassium dihydrogen phosphate, dipotassium orthophosphate, salts of boric acid, citric acid or Tris.

9. The detecting electrode strip according to claim 1, wherein the amount of the buffer solution of the carrier ranges from about 0% to about 6% by weight of the reaction film.

10. The detecting electrode strip according to claim 1, wherein the buffer solution of the carrier has a pH value of from 5.0 to 8.0.

11. The detecting electrode strip according to claim 1, wherein the surfactant of the reaction film is Triton X-100.

12. The detecting electrode strip according to claim 1, wherein the amount of the surfactant of the reaction film ranges from about 2% to about 5% by weight of the reaction film.

13. The detecting electrode strip according to claim 1, wherein the conductive mediator of the reaction film is potassium ferricyanide.

14. The detecting electrode strip according to claim 1, wherein the amount of the conductive mediator of the reaction film ranges from about 2% to about 10% by weight of the reaction film.

15. The detecting electrode strip according to claim 1, which further comprises a protection film covered on the reaction film.

16. A process for producing disposable hemoglobin and hematocrit detecting electrode strip, which comprises:
(a) coating a conducting film on one side of an electric insulating substrate and forming isolated and disconnected an anode and a cathode;
(b) coating an electric insulating film on a part of the conducting film, wherein one end of an uncovered anode of the conducting film is at least a reference electrode and the other end an anode connector, and one end of an uncovered cathode of the conducting film is at least a working electrode and the other end a cathode connector; and
(c) attaching a reaction film to an electric insulating substrate, wherein the reaction film cover a region containing at least the working electrode and the reference electrode so as to connect the working electrode and the reference electrode individually, and wherein the reaction film comprises a carrier, a surfactant and a conductive mediator, the carrier comprises a microcrystalline cellulose, a polymer and a buffer solution; the surfactant comprises an amphoteric substance having a hydrophobic moiety and a hydrophilic moiety; and the conductive mediator comprises an electrolyte with a lower redox potential than that of hemoglobin.

17. The process according to claim 16, wherein is performed by screen printing.

18. The process according to claim 16, wherein the step (c) further comprising the drying at a temperature of 40° C. to 80° C.

19. A hemoglobin and hematocrit detecting equipment, which comprises:
(A) a disposable hemoglobin and hematocrit detecting electrode strip, said strip comprises:
(a) an electric insulating substrate;
(b) a conducting film coated on one side of the electric insulating substrate to form isolated and disconnected an anode and a cathode;
(c) an electric insulating film coated on a part of the conducting film, wherein one end of an uncovered anode of the conducting film forms at least a reference electrode and the other end an anode connector, and one end of an uncovered cathode of the conducting film forms at least a working electrode and the other end a cathode connector; and
(d) a reaction film comprising a carrier, a surfactant and a conductive mediator, being screen printed on a region containing at least the working electrode and the reference electrode so as to connect the working electrode and the reference electrode individually, wherein the carrier comprises a microcrystalline cellulose, a polymer and a buffer solution; the surfactant comprises an amphoteric substance having a hydrophobic moiety and a hydrophilic moiety; and the conductive mediator comprises an electrolyte with a lower redox potential than that of hemoglobin; and
(B) an amperometric sensor, which comprises a voltage output equipment, a signal receiver and a display equipment;
wherein the amperometric sensor is connected to the anode connector and the cathode connector of the hemoglobin and hematocrit detecting electrode strip; the voltage output equipment provides a voltage of below 400 mV to the reaction film of the hemoglobin and hematocrit detecting electrode strip so as to oxidize the conductive mediator from reduction state to oxidation state after being reacted with hemoglobin and hematocrit of the sample; the signal receiver receives a current, voltage or resistance change generated during a redox reaction and transmits the change to the display equipment to display the concentration of hemoglobin and the hematocrit value in the sample.

20. A method for determining hemoglobin and hematocrit, which comprises dropping the liquid sample to the disposable hemoglobin and hematocrit detecting electrode strip according to claim 1, controlling the reaction of hemoglobin and hematocrit at a low operation voltage of below 400 mV and a pH value from 5.0 to 8.0, and determining the concentration of hemoglobin and the hematocrit value in said liquid sample.
